(19) United States
(12) Patent Application Publication
(10) Pub. No.: US 2009/0234241 A1
(43) Pub. Date: Sep. 17, 2009

(54) RECORDING MEDIUM, DEVICE, AND METHOD FOR SLEEP APNEA DETECTION

(75) Inventors: Yasuji OTA, Kawasaki (JP); Kaori Endo, Kawasaki (JP); Takeshi Otani, Kawasaki (JP); Taro Togawa, Kawasaki (JP)

Correspondence Address: Fujitsu Patent Center;C/O CPA Global, P.O. Box 52050, Minneapolis, MN, 55402, US

(73) Assignee: FUJITSU LIMITED, Kawasaki (JP)

(21) Appl. No.: 12/405,624

(22) Filed: Mar. 17, 2009

(30) Foreign Application Priority Data
Mar. 17, 2008 (JP) 2008-068529

(51) Int. Cl. A61B 5/08

(52) U.S. Cl. 600/529

(57) ABSTRACT

A device for sleep apnea detection, includes an external sound recorder that records external sound in environment, a sound/silence determining unit that determines whether an audible sound or no sound is found in the external sound recorded in the external sound recorder, a breathing pace analyzing unit that analyzes, using information of intervals of the audible sound and no sound found by the sound/silence determining unit and the external sound, a breathing pace indicating a cycle of a breathing estimated interval during which breathing is estimated, and an apnea interval extracting unit that extracts a silent interval within the breathing estimated interval based on the breathing pace analyzed by the breathing pace analyzing unit.

### CROSS-REFERENCE TO RELATED APPLICATION

This application is based upon and claims the benefit of priority of the prior Japanese Patent Application No. 2008-068529, filed on Mar. 17, 2008, the entire contents of which are incorporated herein by reference.

### FIELD

The embodiments discussed herein are directed to a recording medium, a device, and a method for sleep apnea detection that detect sleep apnea.

### BACKGROUND

Polysomnography (PSG) has been known as a technology for detecting sleep apnea. The PSG is, for example, used for apnea detection to diagnose sleep apnea syndrome or the like in recent years. In the PSG, electro-oculogram, brain wave, and breathing movement of diaphragm and abdominal muscle are measured for the detection of sleep apnea. Because the PSG requires measurement of electro-oculogram, brain wave, and breathing movement of diaphragm and abdominal muscle, this technology has suffered from restrictions on the place and time of testing.

For this reason, portable apnea detection devices have become known that include pressure sensitive respiratory motion and sound sensors, a recorder, and a data processor. Specifically, such apnea detection devices record, at the recorder, data collected by the respiratory motion sensor and the sound sensor, so as to cause the data processor to calculate index values. Such an apnea detection device is described, for example, in Japanese Patent Application Laid-open No. H10-295695.

As a technology for checking breathing during sleep, Japanese Patent Application Laid-open No. 2004-33254, for example, describes a technology for measuring a breath sound during sleep and visualizing a result of the measured breath sound. The technology allows a user to visually check the visualized breath sound to check sleep apnea.

According to the technology using the portable apnea detection device, the respiratory motion sensor and the sound sensor collect data, the recorder records the collected data, and then the data processor calculates index values. This makes a device configuration relatively large, causing a burden on the user. Further, the technology requires an expert to process the data to detect (diagnose) the sleep apnea, thus causing a cost increase.

According to the technology for measuring a breath sound and visualizing a result of the measured breath sound, a breath sound is merely visualized, and not analyzed. Therefore, an expert has to examine the visualized breath sound to detect (diagnose) the sleep apnea, resulting in a cost increase. Further, a configuration including a checking device is increased in scale, and the cost burden is imposed on the user.

### SUMMARY

It is an object of the present invention to at least partially solve the problems in the conventional technology.

According to one aspect of the present invention, a computer readable recording medium that stores therein a computer program for sleep apnea detection, the computer program causes a computer to execute (a) recording external sound in environment, (b) determining whether an audible sound or no sound is found in the external sound recorded at (a), (c) analyzing, using information of intervals of the audible sound and no sound found at (b) and the external sound, a breathing pace indicating a cycle of a breathing estimated interval during which breathing is estimated, and (d) extracting a silent interval within the breathing estimated interval based on the breathing pace analyzed at (c).

According to another aspect of the present invention, a device for sleep apnea detection, includes an external sound recorder that records external sound in environment, a sound/silence determining unit that determines whether an audible sound or no sound is found in the external sound recorded in the external sound recorder, a breathing pace analyzing unit that analyzes, using information of intervals of the audible sound and no sound found by the sound/silence determining unit and the external sound, a breathing pace indicating a cycle of a breathing estimated interval during which breathing is estimated, and an apnea interval extracting unit that extracts a silent interval within the breathing estimated interval based on the breathing pace analyzed by the breathing pace analyzing unit.

According to still another aspect of the present invention, a method for sleep apnea detection includes recording external sound in environment, determining whether an audible sound or no sound is found in the external sound recorded at the recording, analyzing, using information of intervals of the audible sound and no sound found at the determining and the external sound, a breathing pace indicating a cycle of a breathing estimated interval during which breathing is estimated, and extracting a silent interval within the breathing estimated interval based on the breathing pace analyzed at the analyzing.

Additional objects and advantages of the invention will be set forth in part in the description which follows, and in part will be obvious from the description, or may be learned by practice of the invention. The object and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF DRAWINGS

FIG. 1 is a schematic block diagram of a sleep apnea detection device according to a first embodiment of the present invention;

FIG. 2 depicts an example of a system configuration including the sleep apnea detection device;

FIG. 3 depicts a display example of a result of sleep apnea detection;

FIG. 4 depicts another display example of a result of sleep apnea detection;

FIG. 5 is a graph for explaining a snore determination process;

FIG. 6 is a graph for explaining a breathing pace analysis process;

FIG. 7 is a flowchart of overall processing operations performed by the sleep apnea detection device depicted in FIG. 1;

FIG. 8 is a flowchart of operations performed as an apnea interval determination process by the sleep apnea detection device depicted in FIG. 1; and

FIG. 9 is a schematic diagram of a computer that executes a program for sleep apnea detection.

### DESCRIPTION OF EMBODIMENTS

Exemplary embodiments of a recording medium, a device, and a method for sleep apnea detection according to the present invention will be described in detail with reference to the accompanying drawings.

### [a] First Embodiment

A device for sleep apnea detection according to a first embodiment of the present invention is described regarding its configuration and process flow, followed by advantages achieved in the first embodiment. Specifically, the device for sleep apnea detection is applied to a portable telephone terminal, and sleep apnea is detected by, for example, placing the portable telephone terminal near a user before sleep.

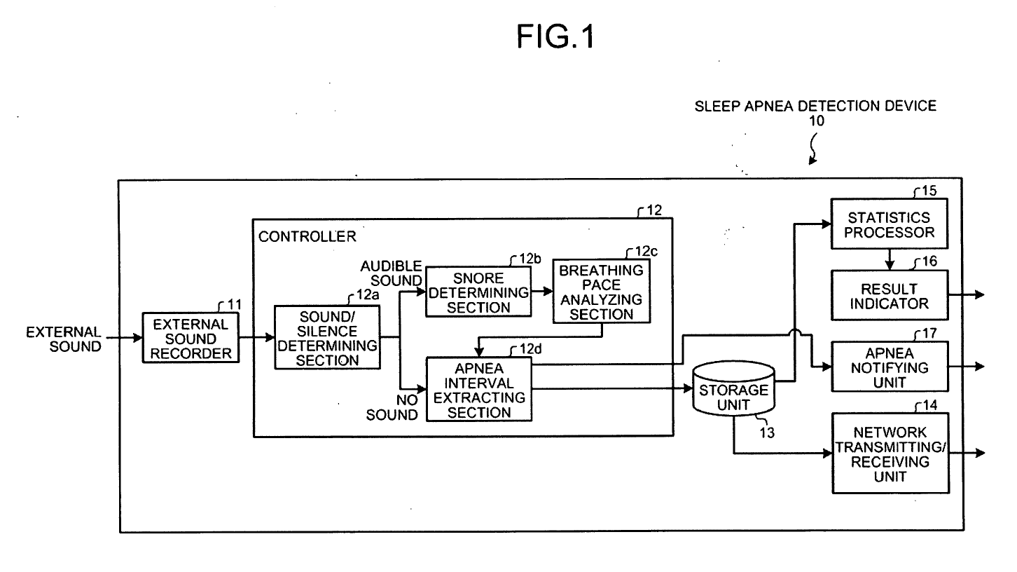

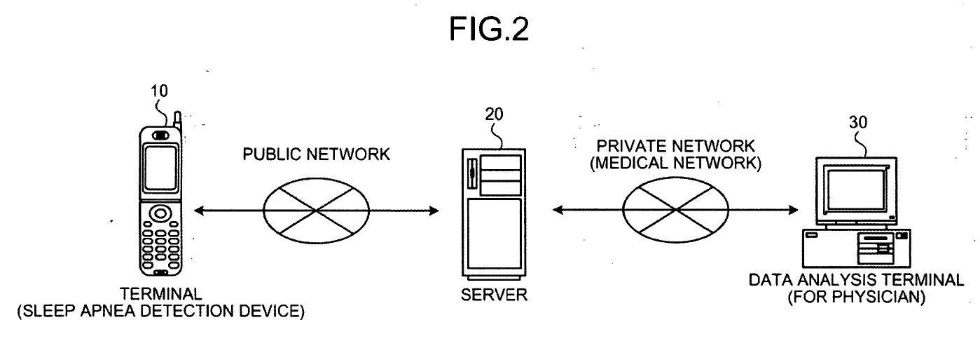

Referring to FIG. 1, a configuration of a sleep apnea detection device 10 according to the first embodiment of the present invention is described. FIG. 1 is a schematic block diagram of the sleep apnea detection device 10. As illustrated in FIG. 1, the sleep apnea detection device 10 includes an external sound recorder 11, a controller 12, a storage unit 13, a network transmitting/receiving unit 14, a statistics processor 15, a result indicator 16, and an apnea notifying unit 17. The sleep apnea detection device 10 is connected to a data analysis terminal 30 via a server 20 (see FIG. 2).

The external sound recorder 11 records external sound in environment in the form of data. The external sound recorder 11 includes a microphone, for example. Specifically, the external sound recorder 11 records the external sound in the form of data, and notifies it to a sound/silence determining section 12a (described later).

The storage unit 13 stores therein a result of extraction of an apnea interval. Specifically, the storage unit 13 receives information of a result of extraction of an apnea interval, performed by an apnea interval extracting section 12d, and stores therein the received information of the extraction result of the apnea interval or the recorded sound.

The network transmitting/receiving unit 14 controls communication of various kinds of information exchanged with the server 20 and the data analysis terminal 30. For example, the network transmitting/receiving unit 14 transmits the extraction result of the apnea interval, stored in the storage unit 13, to the data analysis terminal 30 via the server 20.

Referring to FIG. 2, an example of system configuration including the sleep apnea detection device 10 is described. The system of FIG. 2 includes the sleep apnea detection device 10, and the server 20 and the data analysis terminal 30 both connected to the sleep apnea detection device 10. As illustrated in FIG. 2, the sleep apnea detection device 10 is connected to the server 20 via a public network, and the server 20 is connected to the data analysis terminal 30 (e.g., a terminal used by a physician for diagnosis) via a private network (e.g., a medical network).

Specifically, when the sleep apnea detection device 10 transmits the extraction result of the apnea interval as statistical data to the data analysis terminal 30, a physician can use the extraction result received at the data analysis terminal 30 as auxiliary data for diagnosis. This facilitates diagnosis of the sleep apnea syndrome.

Referring back to FIG. 1, the statistics processor 15 reads out the extraction result of the apnea interval from the storage unit 13, and performs statistical processing. Specifically, the statistics processor 15 reads out the extraction result of the apnea interval from the storage unit 13, performs statistical processing, calculates a risk of the apnea syndrome, and notifies the risk to the result indicator 16. For example, the statistics processor 15 may calculate, as information indicating a risk of the apnea syndrome, a ratio of a time period of the apnea interval with respect to an overall time, a total time of the apnea interval, and the presence or absence of the apnea interval.

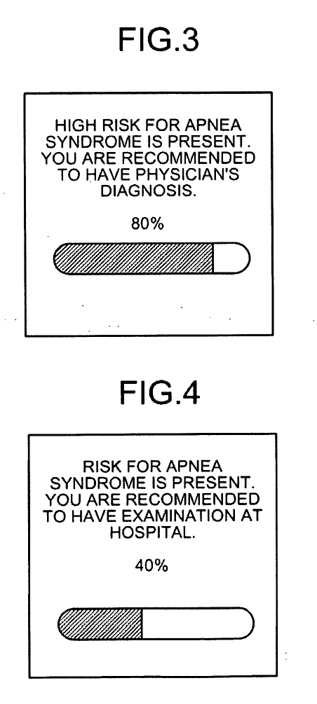

The result indicator 16 indicates to a user a risk of apnea syndrome as a statistical result. The result indicator 16 includes a display or a speaker. For example, the result indicator 16 may indicate a risk of apnea syndrome as a numeric value as depicted in FIGS. 3 and 4, or may display a message for prompting the user to obtain diagnosis from a specialist physician depending on the statistical result. In other words, by indicating to the user a risk of apnea syndrome, the sleep apnea detection device 10 can easily provide the user with a diagnosis result of the apnea syndrome.

For example, when the sleep apnea detection device 10 is applied to a portable telephone terminal, the user may set the portable telephone terminal to detect the sleep apnea, and place it at hand before sleep. In this way, the user can check a diagnosis result of the apnea syndrome after waking from sleep.

The apnea notifying unit 17 notifies an apnea condition to the user when an abruptly occurred silent interval is extracted from a breathing estimated interval. The apnea notifying unit 17 includes an alarm or a vibrator. Specifically, upon receiving the notification of the apnea condition from the apnea interval extracting section 12d (described later), the apnea notifying unit 17 actuates the alarm or the vibrator.

Specifically, the apnea condition is notified to the user during sleep, so that the apnea is prevented. As to whether the apnea condition is notified, the user may also set the alarm or the vibrator not to operate.

The controller 12 includes an internal memory that stores therein computer programs specifying various processing procedures and predetermined data, and performs various processes using these programs and data. In the first embodiment, the controller 12 includes the sound/silence determining section 12a, a snore determining section 12b, a breathing pace analyzing section 12c, and the apnea interval extracting section 12d, as elements involved in the present invention.

The sound/silence determining section 12a determines at a predetermined time interval whether an audible sound or no sound is in external sound. Specifically, the sound/silence determining section 12a acquires data of the external sound recorded by the external sound recorder 11, and divides the data by frame of a predetermined time interval. The sound/silence determining section 12a then analyzes audio characteristics in each frame, and determines whether an audible sound or no sound is found. If an audible sound is found, the sound/silence determining section 12a notifies the snore determining section 12b that the audible sound is found, and also notifies of the data of the external sound.

If no sound is found, the sound/silence determining section 12a notifies the apnea interval extracting section 12d that no sound is found, and also notifies of the data of the external sound. The silent state may include completely no sound, or may include only a minor sound that is equal to or less than a predetermined threshold (e.g., noise).

The snore determining section 12b determines whether snoring is included in the data of the external sound in an audible-sound interval. Specifically, the snore determining section 12b analyzes characteristics of the data of the external sound notified by the sound/silence determining section 12a, so as to determine whether a snoring sound is found. If a snoring sound is found, the snore determining section 12b extracts a snoring interval, and notifies it to the breathing pace analyzing section 12c.

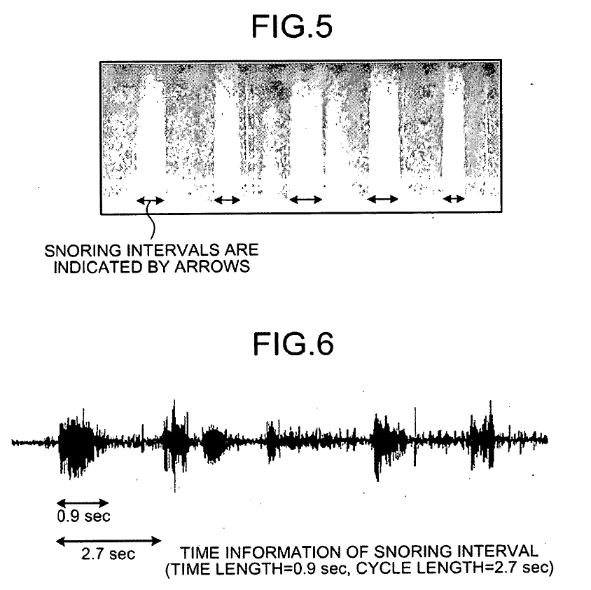

Referring to FIG. 5, a specific example of a snore determination process is described. FIG. 5 depicts spectral characteristics of the external sound, indicated as frequency distribution information. As depicted in FIG. 5, the snore determining section 12b analyzes the characteristics of the data of the external sound, determines a snoring sound based on a specific pattern indicating a snore, and extracts time periods indicated by arrows as snoring intervals.

Using information of intervals of the audible sound and no sound, the breathing pace analyzing section 12c analyzes a breathing pace indicating a cycle of a “snoring estimated interval” during which the use is estimated to be breathing (snoring). Specifically, the breathing pace analyzing section 12c calculates a time length and a cycle length of the snoring interval notified by the snore determining section 12b, so as to analyze the user's breathing pace also using the information of the silent intervals. The breathing pace analyzing section 12c then notifies information of the breathing pace and information of the time length of the snoring interval to the apnea interval extracting section 12d.

Referring to FIG. 6, a specific example of a breathing pace analysis process is described. As illustrated in FIG. 6, the breathing pace analyzing section 12c calculates “0.9 second” as a time length of a snoring interval and “2.7 seconds” as a cycle length of the snoring interval. The time length and the cycle length may be calculated as mean values in a predetermined time period to ensure statistical accuracy of the values.

As the breathing pace analysis process, the breathing pace analyzing section 12c calculates a breathing pace based on a ratio of a snoring interval with respect to a certain time period (“one minute” in the example depicted in FIG. 6) using an equation (1):

$${Freq}_{ave} = \frac{60 U}{{P}_{ave}} \qquad ( 1 )$$

In the equation (1), “Pₐᵥₑ” is an averaged cycle length of snoring intervals in one second, and “Freqₐᵥₑ” is a breathing pace in one minute.

The apnea interval extracting section 12d extracts an abruptly occurred silent interval from the breathing estimated interval based on the analyzed breathing pace. Specifically, the apnea interval extracting section 12d receives data of the external sound from the sound/silence determining section 12a, and receives information of the breathing pace and information of the time length of the snoring interval from the breathing pace analyzing section 12c.

The apnea interval extracting section 12d acquires information of the snoring estimated interval based on the breathing pace and the time length of the snoring interval, so as to determine whether the silent interval during which no sound is found is within the snoring estimated interval, i.e., whether the silent interval is disruption of snoring (breathing) in the snoring estimated interval.

If the silent interval is disruption of snoring in the snoring estimated interval, the apnea interval extracting section 12d measures a time of the disruption and its duration, and compares with the result of the breathing pace analysis. In this way, the apnea interval extracting section 12d determines whether the disruption of snoring is apnea, or just disappearance of snoring.

If the disruption of snoring is apnea, the apnea interval extracting section 12d extracts, as an apnea interval, the silent interval from the snoring estimated interval. The apnea interval extracting section 12d then notifies the apnea condition to the apnea notifying unit 17, and also notifies of an extraction result of the apnea interval to the storage unit 13. If the disruption of snoring is not apnea, the sleep apnea detection device 10 determines that the disruption is not apnea, but just disappearance of snoring.

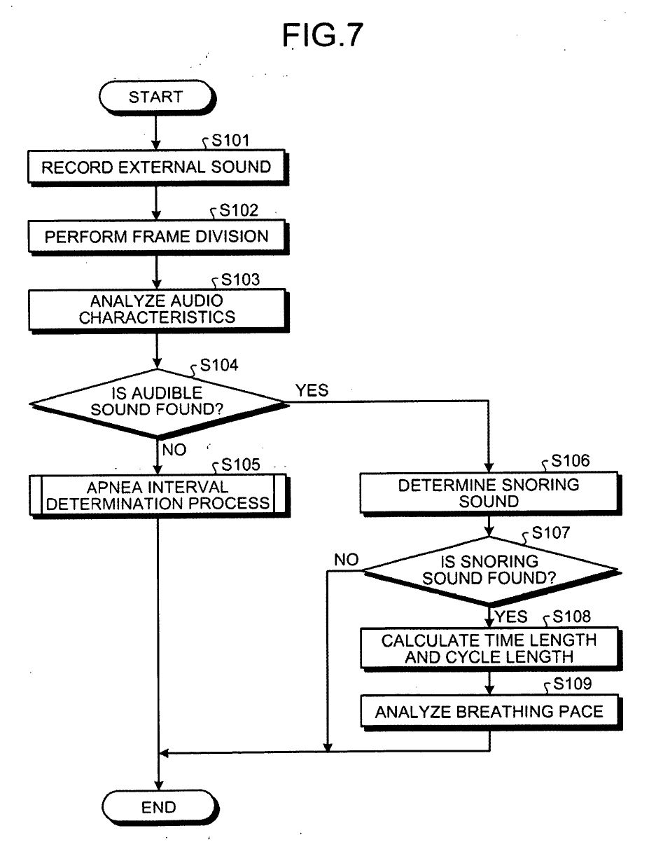

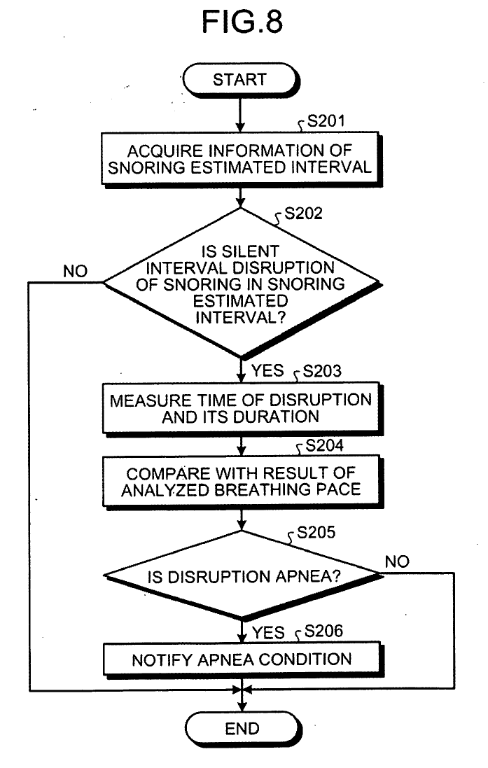

Referring to FIGS. 7 and 8, processes performed by the sleep apnea detection device 10 according to the first embodiment are described. FIG. 7 is a flowchart of overall processing operations performed by the sleep apnea detection device 10 according to the first embodiment. FIG. 8 is a flowchart of operations performed as an apnea interval process by the sleep apnea detection device 10 according to the first embodiment.

As illustrated in FIG. 7, the sleep apnea detection device 10 records external sound at the external sound recorder 11 in the form of data (step S101), divides the data of the recorded external sound by frame at a predetermined time interval (step S102), analyzes audio characteristics of the data, and determines whether an audible sound or no sound is found in the external sound (step S103).

If an audible sound is found (YES at step S104), the sleep apnea detection device 10 analyzes, based on the characteristics of the external sound, whether the external sound is a snoring sound (step S106), so as to determine whether a snoring sound is found (step S107). If a snoring sound is found (YES at step S107), the sleep apnea detection device 10 calculates a time length and a cycle length of the snoring interval (step S108), and analyzes the user's breathing pace also using the information including silent intervals (step S109).

Referring back to step S104, if no sound is found (NO at step S104), the sleep apnea detection device 10 performs an apnea interval determination process (described in detail later referring to FIG. 8) (step S105).

Referring to FIG. 8, an apnea interval determination process performed by the sleep apnea detection device 10 is described. As illustrated in FIG. 8, the sleep apnea detection device 10 acquires information of the snoring estimated interval based on the analyzed breathing pace and the time length of the snoring interval (step S201) so as to determine whether the silent interval during which no sound is found is within the snoring estimated interval, i.e., whether the silent interval is disruption of snoring (breathing) in the snoring estimated interval (step S202).

If the silent interval is not disruption of snoring in the snoring estimated interval (NO at step S202), the sleep apnea detection device 10 terminates the apnea interval determination process. On the contrary, if the silent interval is disruption of snoring in the snoring estimated interval (YES at step S202), the sleep apnea detection device 10 measures a time of the disruption and its duration (step S203), compares with the result of the breathing pace analysis (step S204), and determines whether the disruption of snoring is apnea, or just disappearance of snoring (step S205).

If the disruption of snoring is apnea (YES at step S205), the sleep apnea detection device 10 notifies the apnea condition to the user (step S206), and terminates the process. On the contrary, if the disruption of snoring is not apnea (NO at step S205), the sleep apnea detection device 10 terminates the process, regarding the silent interval as just disappearance of snoring, not apnea.

As described above, by detecting sleep apnea using the external sound, the sleep apnea detection device 10 detects apnea without using sensors other than the external sound recorder 11 (e.g., microphone) that records the external sound. This enables detection of sleep apnea with simple operation and low cost, without requiring large-scale equipment and an examination by a physician.

According to the first embodiment, if an audible sound is found, the sleep apnea detection device 10 determines whether snoring is included in an audible-sound interval during which the audible sound is found. If snoring is included, the sleep apnea detection device 10 analyzes a breathing pace using a snoring interval during which the snoring is included. As such, snoring is determined through analysis of audio characteristics of the external sound, and a breathing pace is analyzed using the snoring. This enables apnea detection without using sensors other than the external sound recorder 11 (e.g., microphone) that records the external sound. Further, the snoring can be determined easily because a snoring sound is larger than breathing during sleep and has a typical sound made by air coming off from the nose.

According to the first embodiment, the sleep apnea detection device 10 calculates a cycle length of a snoring interval, and analyzes a breathing pace using the cycle length of the snoring interval. Thus, a breathing pace can be analyzed easily based on a cycle of snoring.

According to the first embodiment, if a silent interval is extracted, the sleep apnea detection device 10 determines, using calculated cycle information of the snoring interval, whether the silent interval is disruption of snoring due to apnea, or disappearance of snoring. Because determination can be made whether the silent interval is just disappearance of snoring, not due to apnea, it is possible to detect sleep apnea with improved accuracy.

According to the first embodiment, if a silent interval within the breathing estimated interval is extracted, the sleep apnea detection device 10 notifies the apnea condition to the user. This allows the sleeping user to be notified of the apnea condition, thus preventing the apnea.

According to the first embodiment, the sleep apnea detection device 10 performs statistical processing on an extraction result of a silent interval extracted, calculates a risk of apnea syndrome, and indicates the calculated risk of apnea syndrome on an indicator. Thus, a diagnosis result of apnea syndrome can be easily provided to the user.

According to the first embodiment, the sleep apnea detection device 10 performs statistical processing on an extraction result of a silent interval extracted, calculates a risk of apnea syndrome, and outputs the calculated risk of apnea syndrome to an external terminal for medical use. This enables health care service coupling the data with knowledge of specialist physicians.

According to the first embodiment, the sleep apnea detection device 10 achieves a function of preventing apnea in conjunction with the alarm and the vibrator, and provides health care service utilizing knowledge of specialist physicians by storing data in the server via the network. All the hard components mentioned above are installed in commercially available portable telephones, and therefore, the above function is realized by software of the portable telephones, without the need of buying and installing an additional dedicated sensor.

This brings about an advantage as not imposing a heavy cost burden on the user. Further, stable data can be recorded in a normal living environment, in contrast to testing performed by a special engineer in a place where special equipment is installed.

### [b] Second Embodiment

The present invention may be practiced in different embodiments other than the first embodiment. An alternative embodiment is described as a second embodiment of the present invention.

### [b-1] Sleep Breathing

In the first embodiment, a breathing pace is analyzed by determining whether “snoring” is included in data of external sound within an audible-sound interval. The present invention is not limited to this arrangement, and a breathing pace may be analyzed by determining whether “sleep breathing” is included in data of external sound in an audible-sound interval.

Specifically, a sleep apnea detection device according to the second embodiment determines whether sleep breathing is included in the external sound. When a sleep breathing interval is extracted, the sleep apnea detection device analyzes a breathing pace using the sleep breathing interval, instead of the snoring interval, and extracts an apnea interval as in the first embodiment. A breathing pace may be analyzed based on any sound related to the user's breathing, as well as snoring and sleep breathing.

As such, the sleep apnea detection device analyzes a breathing pace by extracting a sleep breathing interval. Thus, users who do not snore can also detect sleep apnea.

### [b-2] System Configuration

Constituting elements of the devices illustrated in the drawings represent ideational functions, and their physical arrangements are not necessarily the same as those depicted in the drawings. Specifically, the arrangement of distributing and integrating the elements is not limited to those specifically depicted in the drawings, and all of or any portion of the devices can be distributed and integrated concerning functional and physical aspects based on given units, with loads on the devices and use conditions taken into account. For example, the sound/silence determining section 12a and the snore determining section 12b may be integrated.

### [b-3] Computer Programs

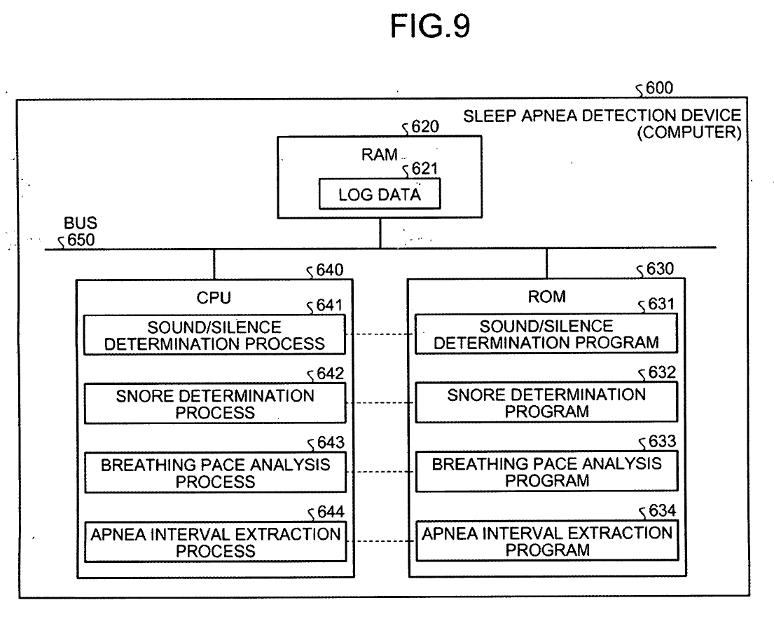

The various processes described in the embodiments can be performed with a computer that executes computer programs prepared in advance. Referring to FIG. 9, an example of a computer that executes programs performing the same functions as those in the embodiments is described. FIG. 9 is a schematic diagram of a computer that executes sleep apnea detection programs.

As illustrated in FIG. 9, a computer 600 serving as a sleep apnea detection device includes a random access memory (RAM) 620, a read only memory (ROM) 630, and a central processing unit (CPU) 640, all connected via a bus 650.

The ROM 630 stores therein programs for sleep apnea detection, which perform the same functions as those described in the above embodiments. Specifically, a sound/silence determination program 631, a snore determination program 632, a breathing pace analysis program 633, and an apnea interval extraction program 634 are stored in advance, as depicted in FIG. 9. Such computer programs 631 to 634 may be integrated or distributed appropriately, as in the constituting elements of the sleep apnea detection device 10 depicted in FIG. 1.

The CPU 640 reads out the computer programs 631 to 634 from the ROM 630 and executes them, so that the computer programs 631 to 634 serve to perform a sound/silence determination process 641, a snore determination process 642, a breathing pace analysis process 643, and an apnea interval extraction process 644, as depicted in FIG. 9. The processes 641 to 644 correspond to the sound/silence determining section 12a, the snore determining section 12b, the breathing pace analyzing section 12c, and the apnea interval extracting section 12d depicted in FIG. 1.

The CPU 640 stores in the RAM 620 results of the sleep apnea detection process as log data 621. The CPU 640 performs a predetermined process based on the log data 621 stored in the RAM 620, or transmits the log data 621 to an external terminal.

A computer program according to the embodiment detects sleep apnea using external sound. This enables detection of sleep apnea without requiring large-scale equipment and an examination by a physician. Thus, sleep apnea detection is advantageously realized with simple operation and low cost.

All examples and conditional language recited herein are intended for pedagogical purposes to aid the reader in understanding the principles of the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present invention has been described in detail, it should be understood that the various changes, substitutions, and alterations could be made hereto without departing from the spirit and scope of the invention.

What is claimed is:

1. A computer readable recording medium that stores therein a computer program for sleep apnea detection, the computer program causing a computer to execute:
(a) recording external sound in environment;
(b) determining whether an audible sound or no sound is found in the external sound recorded at (a);
(c) analyzing, using information of intervals of the audible sound and no sound found at (b) and the external sound, a breathing pace indicating a cycle of a breathing estimated interval during which breathing is estimated; and
(d) extracting a silent interval within the breathing estimated interval based on the breathing pace analyzed at (c).

2. The computer readable recording medium according to claim 1, further causing the computer to execute
(e) determining, when the audible sound is found at (b), whether snoring or sleep breathing is included in an audible-sound interval during which the audible sound is found, wherein
when the snoring or the sleep breathing is determined to be included at (e), (c) includes analyzing the breathing pace by using one of a snoring interval and a sleep breathing interval during which the snoring and the sleep breathing are found respectively.

3. The computer readable recording medium according to claim 2, wherein (c) includes calculating a cycle length of one of the snoring interval and the sleep breathing interval, so as to analyze the breathing pace using the cycle length.

4. The computer readable recording medium according to claim 3, wherein, when the silent interval within the breathing estimated interval is extracted, (d) includes determining, using cycle information of the snoring interval calculated at (c), whether the silent interval is disruption of snoring due to apnea, or disappearance of snoring.

5. The computer readable recording medium according to claim 1, further causing the computer to execute (f) notifying apnea condition to a user, when the silent interval within the breathing estimated interval is extracted at (d).

6. The computer readable recording medium according to claim 1, further causing the computer to execute
(g) performing statistical processing on an extraction result of the silent interval extracted at (d), so as to calculate a risk of apnea syndrome; and
(h) displaying the risk of apnea syndrome calculated at (g) on a display unit.

7. The computer readable recording medium according to claim 1, further causing the computer to execute
(g) performing statistical processing on an extraction result of the silent interval extracted at (d), so as to calculate a risk of apnea syndrome; and
(i) outputting the risk of apnea syndrome calculated at (g) to an external terminal for medical use.

8. A device for sleep apnea detection, comprising:
an external sound recorder that records external sound in environment;
a sound/silence determining unit that determines whether an audible sound or no sound is found in the external sound recorded in the external sound recorder;
a breathing pace analyzing unit that analyzes, using information of intervals of the audible sound and no sound found by the sound/silence determining unit and the external sound, a breathing pace indicating a cycle of a breathing estimated interval during which breathing is estimated; and
an apnea interval extracting unit that extracts a silent interval within the breathing estimated interval based on the breathing pace analyzed by the breathing pace analyzing unit.

9. A method for sleep apnea detection, comprising:
recording external sound in environment;
determining whether an audible sound or no sound is found in the external sound recorded at the recording;
analyzing, using information of intervals of the audible sound and no sound found at the determining and the external sound, a breathing pace indicating a cycle of a breathing estimated interval during which breathing is estimated; and
extracting a silent interval within the breathing estimated interval based on the breathing pace analyzed at the analyzing.
